SEQUENCE LISTING (4 sequences)

SEQ ID NO: 1 - protein, 173 aa, Homo sapiens
TPLGPASSLPQSFLLKCLEQVRKIQGDGAALQEKLATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 173 aa, Artificial Sequence
  recombinant human G-CSF analog
TPLGPASSLPQSFLLKSLEQVRKIQGDGAALQEKLCATYKLCHPEELVLLGHSLGIPWAPLSSCSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 221 aa, Artificial Sequence
  Human Immunoglobulin Fc fragment
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 221 aa, Artificial Sequence; identical to SEQ ID NO: 3
  Human Immunoglobulin Fc fragment

(19) United States
(12) Patent Application Publication
(10) Pub. No.: US 2021/0169847 A1
(43) Pub. Date: Jun. 10, 2021

(54) METHODS OF TREATING CHEMOTHERAPY OR RADIOTHERAPY INDUCED NEUTROPENIA

(71) Applicant: HANMI PHARM. CO., LTD., Gyeonggi-do (KR)

(72) Inventors: Jae Hyuk CHOI, Gyeonggi-do (KR); Eun Jung KIM, Gyeonggi-do (KR); Yu Yon KIM, Gyeonggi-do (KR); Gyu Hyan LEE, Gyeonggi-do (KR); Hyesun HAN, Gyeonggi-do (KR)

(21) Appl. No.: 17/113,571

(22) Filed: Dec. 7, 2020

Related U.S. Application Data
(60) Provisional application No. 62/944,359, filed on Dec. 5, 2019.

(51) Int. Cl. A61K 31/40; A61P 7/00

(52) U.S. Cl. CPC A61K 31/40; A61K 45/06; A61P 7/00

(57) ABSTRACT

A method of treating chemotherapy-induced neutropenia in a patient in need thereof according to an embodiment of the present disclosure includes administering to the patient an effective amount of Eflapegrastim. A method of treating radiation-induced neutropenia in a patient in need thereof according to an embodiment of the present disclosure includes administering to the patient an effective amount of Eflapegrastim.

### CROSS-REFERENCE TO RELATED APPLICATION(S)

This application claims the benefit under 35 USC § 119(a) of US Provisional Patent Application No. 62/944,359 filed on Dec. 5, 2019, the entire disclosure of which is incorporated herein by reference for all purposes.

### TECHNICAL FIELD

The present invention relates to pharmaceutical compositions comprising protein complexes, and medical uses thereof for treating or preventing a condition characterized by compromised white blood cell production, such as neutropenia. The protein complex can be formed by linking an immunoglobulin Fc region to a physiologically active polypeptide via a non-peptidyl polymer, in which the non-peptidyl polymer is linked to the immunoglobulin Fc region.

### BACKGROUND

Human granulocyte-colony stimulating factor (G-CSF) is a hematopoietic glycoprotein produced by stromal cells, macrophages, endothelial cells, fibroblasts and monocytes. The G-CSF binds with high affinity to the G-CSF receptor expressed on neutrophilic precursor cells in the bone marrow and induce them to proliferate and differentiate into infection fighting neutrophils without significant haemopoietic effects on other lineages of blood cells. The use of recombinant G-CSF preparations is a well-established treatment for accelerating bone marrow recovery, for preventing the onset of severe myelosuppression and its correlated complications and for reducing febrile neutropenia (FN) in patients with non-myeloid malignancies under radio or chemotherapies.

Pegfilgrastim (NEULASTA®; Amgen Inc.) is the most popular PEGylated form of the recombinant human G-CSF. Eflapegrastim is a long-acting G-CSF that has been developed to reduce the severity and duration of severe neutropenia, as well as complications of neutropenia, associated with the use of myelosuppressive anti-cancer drugs. At present, the recommended dosing regimen for both Eflapegrastim (ROLONTIS®, HM10460A) and Pegfilgrastim is next day administration following cytotoxic chemotherapy, which requires patients typically in a weakened and uncomfortable state after undergoing chemotherapy, to travel to the hospital again.

Therefore there is an unmet need to develop a same day dosing regimen for a long-acting G-CSF that eases patient burden while providing comparable or superior efficacy in the treatment of neutropenia.

### SUMMARY

In one aspect, provided herein are methods for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient is administered a chemotherapeutic agent.

In another aspect, provided herein are methods for treating or preventing the condition characterized by compromised white blood cell production in a patient in need thereof, comprising administering an effective amount of Eflapegrastim within a period of less than 24 hours after the patient is administered a chemotherapeutic agent.

In another aspect, provided herein are methods for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient receives radiotherapy.

In another aspect, provided herein are methods for treating or preventing the condition characterized by compromised white blood cell production in a patient in need thereof, comprising administering an effective amount of Eflapegrastim within a period of less than 24 hours after the patient receives a radiotherapy.

In some embodiments, the condition characterized by compromised white blood cell production is selected from the group consisting of: chemotherapy-induced neutropenia, radiotherapy-induced neutropenia, reduced hematopoietic function, reduced immune function, reduced neutrophil count, reduced neutrophil mobilization, mobilization of peripheral blood progenitor cells, sepsis, bone marrow transplants, infectious diseases, leucopenia, thrombocytopenia, anemia, enhancing engraftment of bone marrow during transplantation, enhancing bone marrow recovery in treatment of radiation, chemical or chemotherapeutic induced bone marrow aplasia or myelosuppression, radiotherapy-induced bone marrow aplasia or myelosuppression, and acquired immune deficiency syndrome.

In some embodiments, the condition characterized by compromised white blood cell production is a chemotherapy-induced neutropenia or a radiotherapy-induced neutropenia.

In some embodiments, the method reduces the duration of chemotherapy-induced neutropenia or radiotherapy-induced neutropenia in a patient in need thereof.

In some embodiments, the method comprises administering an effective amount of Eflapegrastim on the same day when the patient is administered a chemotherapeutic agent or a radiotherapy.

In some embodiments, administering the effective amount of Eflapegrastim reduces the duration of an absolute neutrophil count of less than about 0.5×10⁹/L in the patient less than about 6 hours, about 12 hours, or 24 hours.

In some embodiments, administering the effective amount of Eflapegrastim prevents the absolute neutrophil count in the patient from reaching less than about 0.5×10⁹/L.

In some embodiments, upon administration of the effective amount of Eflapegrastim, an absolute neutrophil count of the patient may increase from the first occurrence of less than about 0.5×10⁹/L to greater than or equal to about 1.5×10⁹/L within less than about four days, about seven days, or about ten days.

### BRIEF DESCRIPTION OF THE DRAWINGS

FIG. 1 shows the results of chromatography of an immunoglobulin Fc fragment obtained by cleavage of an immunoglobulin with papain.

FIG. 2 shows the results of SDS-PAGE of a purified immunoglobulin Fc fragment (M: molecular size marker, lane 1: IgG, lane 2: Fc).

FIG. 3 shows the effects of HM10460A and Pegfilgrastim on absolute neutrophil count (ANC) following acute TC induced neutropenia in normal SD rats. 0 hr (A), +2 hr (B), +5 hr (C), and +24 hr (D) after chemotherapy.

### DETAILED DESCRIPTION

As generally described herein, the present disclosure provides methods for increasing the absolute neutrophil count, the number of granulocytes in a subject eligible for a bone marrow transplant, stem cell production, hematopoiesis, the number of hematopoietic progenitor cells, or stem cell production in a donor, or for treating or preventing the condition characterized by compromised white blood cell production in a patient in need thereof, comprising administering an effective amount of Eflapegrastim within a period of less than 24 hours after the patient is administered a chemotherapeutic agent or receives a radiotherapy.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments is variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, the term “severe neutropenia” is defined as neutropenia having an absolute neutrophil count less than 0.5×10⁹/L. The terms “severe neutropenia” and “Grade 4 neutropenia” may be used interchangeably.

As used herein, “pharmaceutical composition” or “pharmaceutical formulation” refers to the combination of an active agent with a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, radiotherapy, or treatment for a neurodegenerative disease). Eflapegrastim is administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound refers to an amount sufficient to elicit the desired biological response, e.g., to treat upper tract urothelial carcinoma or non-muscle invasive bladder cancer. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

The terms “protein conjugate” or “conjugate”, as used herein, refer to a compound comprising one or more physiologically active polypeptides, one or more non-peptide polymers having a reactive group at both ends and one or more immunoglobulin Fc fragments, wherein the three components are covalently linked. In addition, to be distinguished from the “conjugate”, a construct comprising only two different molecules selected from a physiologically active polypeptide, a non-peptide polymer and an immunoglobulin Fc fragment, wherein the two molecules are covalently linked together, is designated as a “complex”.

The term “immunoglobulin Fc fragment”, as used herein, refers to a protein that contains the heavy-chain constant region 2 (C_{H}2) and the heavy-chain constant region 3 (C_{H}3) of an immunoglobulin, and not the variable regions of the heavy and light chains, the heavy-chain constant region 1 (C_{H}1) and the light-chain constant region 1 (C_{L}I) of the immunoglobulin. It may further include the hinge region at the heavy-chain constant region. Also, the immunoglobulin Fc fragment of the present invention may contain a portion or all of the heavy-chain constant region 1 (C_{H}1) and/or the light-chain constant region 1 (C_{L}1), except for the variable regions of the heavy and light chains. Also as long as it has a physiological function substantially similar to or better than the native protein the IgG Fc fragment is a fragment having a deletion in a relatively long portion of the amino acid sequence of C_{H}2 and/or C_{H}3. That is, the immunoglobulin Fc fragment of the present invention may comprise 1) a C_{H}1 domain, a C_{H}2 domain, a C_{H}3 domain and a C_{H}4 domain, 2) a C_{H}1 domain and a C_{H}2 domain, 3) a C_{H}1 domain and a C_{H}3 domain, 4) a C_{H}2 domain and a C_{H}3 domain, 5) a combination of one or more domains and an immunoglobulin hinge region (or a portion of the hinge region), and 6) a dimer of each domain of the heavy-chain constant regions and the light-chain constant region.

As used herein, the term “deglycosylation” refers to enzymatically remove sugar moieties from an Fc fragment, and the term “aglycosylation” means that an Fc fragment is produced in an unglycosylated form by a prokaryote, preferably E. coli.

The term “combination”, as used herein, means that polypeptides encoding single-chain immunoglobulin Fc regions of the same origin are linked to a single-chain polypeptide of a different origin to form a dimer or multimer. That is, a dimer or multimer is formed from two or more fragments selected-from the group consisting of IgG1 Fc, IgG2 Fc, IgG3 Fc and IgG4 Fc fragments.

The term “hybrid”, as used herein, means that sequences encoding two or more immunoglobulin Fc fragments of different origin are present in a single-chain immunoglobulin Fc fragment.

The term “non-peptide polymer”, as used herein, refers to a biocompatible polymer including two or more repeating units linked to each other by a covalent bond excluding the peptide bond.

The terms “physiologically active polypeptide”, “physiologically active protein”, “active polypeptide”, “polypeptide drug” or “protein drug”, as used herein, are interchangeable in their meanings, and are featured in that they are in a physiologically active form exhibiting various in vivo physiological functions.

### Eflapegrastim

Eflapegrastim, as known as ROLONTIS®, SPI-2012, HM10460A, and ^{17,65}S-G-CSF, is a long-acting granulocyte-colony stimulating factor (G-CSF) that has been developed to reduce the severity and duration of severe neutropenia, as well as complications of neutropenia, associated with the use of myelosuppressive anti-cancer drugs or radiotherapy. Eflapegrastim consists of a recombinant human G-CSF analog (ef-G-CSF) and a recombinant fragment of the Fc region of human immunoglobulin G4 (IgG4), linked by a Bifunctional polyethylene glycol linker. In certain embodiments, the recombinant human G-CSF analog (ef-G-CSF) varies from human G-CSF (SEQ ID NO: 1) at positions 17 and 65 which are substituted with serine (SEQ ID NO: 2). Without wishing to be bound by theory, it is believed that the Fc region of human IgG4 increases the serum half-life of ef-G-CSF.

ef-G-CSF is produced by transformed E. coli in soluble form in the periplasmic space. Separately, the Fc fragment is produced in transformed E. coli as an inclusion body. The ef-G-CSF and the Fc fragment are independently isolated and purified through successive purification steps. The purified ef-G-CSF (SEQ ID NO: 2) and Fc fragment (SEQ ID NOs: 3 and 4) are then linked via a 3.4 kDa PEG molecule that was designed with reactive groups at both ends. Eflapegrastim itself is the molecule resulting from the PEG linker binding at each of the N-termini of ef-G-CSF and the Fc fragment. The G-CSF analog is conjugated to the 3.4 kDa polyethylene glycol analogue with propyl aldehyde end groups at both ends, (OHCCH₂CH₂(OCH₂CH₂)nOCH₂CH₂CHO) at the nitrogen atom of its N-terminal The residue via reductive amination to form a covalent bond. The resulting G-CSF-PEG complex is then linked to the N-terminal Pro at the nitrogen of the recombinant Fc fragment variant produced in E. coli via reductive amination to yield the final conjugate of Eflapegrastim.

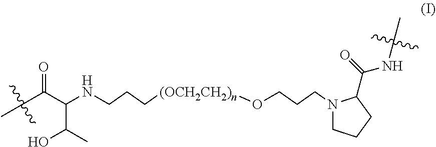

In one aspect, provided herein is Eflapegrastim, for use in the method for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient is administered a chemotherapeutic agent.

In another aspect, provided herein is Eflapegrastim, for use in the treatment or prevention of the condition characterized by compromised white blood cell production in a patient in need thereof, comprising administering an effective amount of Eflapegrastim within a period of less than 24 hours after the patient is administered a chemotherapeutic agent.

In another aspect, provided herein is Eflapegrastim, for use in the method for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient receives radiotherapy.

In another aspect, provided herein is Eflapegrastim, for use in the in the treatment or prevention of the condition characterized by compromised white blood cell production in a patient in need thereof, comprising administering an effective amount of Eflapegrastim within a period of less than 24 hours after the patient receives a radiotherapy.

The details described below in the sections Treatment of Chemotherapy Induced Neutropenia and Treatment of Radiotherapy-Induced Neutropenia may be applied to Eflapegrastim here.

### Pharmaceutical Compositions

In one aspect, provided herein is a pharmaceutical composition comprising Eflapegrastim, and a pharmaceutically acceptable carrier, for use in the method for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient is administered a chemotherapeutic agent.

In another aspect, provided herein is a pharmaceutical composition comprising Eflapegrastim, and a pharmaceutically acceptable carrier, for use in the treatment or prevention of the condition characterized by compromised white blood cell production in a patient in need thereof, comprising administering an effective amount of Eflapegrastim within a period of less than 24 hours after the patient is administered a chemotherapeutic agent.

In one aspect, provided herein is a pharmaceutical composition comprising Eflapegrastim, and a pharmaceutically acceptable carrier, for use in the method for increasing the absolute neutrophil count, the number of granulocytes in a subject eligible for a bone marrow transplant, stem cell production, hematopoiesis, the number of hematopoietic progenitor cells, or stem cell production in a donor in a patient in need thereof, comprising administering an effective amount of Eflapegrastim within a period of less than 24 hours after the patient receives radiotherapy.

In another aspect, provided herein is a pharmaceutical composition comprising Eflapegrastim, and a pharmaceutically acceptable carrier, for use in the treatment or prevention of the condition characterized by compromised white blood cell production in a patient in need thereof, comprising administering an effective amount of Eflapegrastim within a period of less than 24 hours after the patient receives a radiotherapy.

In certain embodiments, the pharmaceutically acceptable carrier is a phosphate buffered saline. In some embodiments, the phosphate buffered saline is Dulbecco's phosphate buffered saline. In certain embodiments, the pharmaceutically acceptable carrier is a citrate buffer.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical compositions disclosed herein are administered parenterally. In some embodiments pharmaceutical compositions disclosed herein are administered by subcutaneous administration.

The pharmaceutical compositions provided herein is presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a subcutaneous injection solution.

In certain embodiments, the compounds provided herein can be administered as the sole active agent, or they can be administered in combination with other active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

The details described below in the sections Treatment of Chemotherapy Induced Neutropenia and Treatment of Radiotherapy-Induced Neutropenia may be applied to Pharmaceutical Compositions here.

### Methods of Use and Treatment

### Treatment of Chemotherapy-Induced Neutropenia

In one aspect, provided herein is a method for increasing the absolute neutrophil count, the number of granulocytes in a subject eligible for a bone marrow transplant, stem cell production, hematopoiesis, the number of hematopoietic progenitor cells, or stem cell production in a donor, comprising administering an effective amount of Eflapegrastim within a period of less than 24 hours after the patient is administered a chemotherapeutic agent.

In another aspect, provided herein is a method for treating or preventing the condition characterized by compromised white blood cell production in a patient in need thereof, comprising administering an effective amount of Eflapegrastim within a period of less than 24 hours after the patient is administered a chemotherapeutic agent.

In some embodiments, the condition characterized by compromised white blood cell production is selected from the group consisting of: chemotherapy-induced neutropenia, radiotherapy-induced neutropenia, reduced hematopoietic function, reduced immune function, reduced neutrophil count, reduced neutrophil mobilization, mobilization of peripheral blood progenitor cells, sepsis, bone marrow transplants, infectious diseases, leucopenia, thrombocytopenia, anemia, enhancing engraftment of bone marrow during transplantation, enhancing bone marrow recovery in treatment of radiation, chemical or chemotherapeutic induced bone marrow aplasia or myelosuppression, and acquired immune deficiency syndrome.

In an embodiment, the condition is a chemotherapy-induced neutropenia. In an embodiment, the method may reduce the duration of chemotherapy-induced neutropenia in a patient in need thereof.

In an embodiment, the method comprises administering an effective amount of Eflapegrastim on the same day when the patient is administered a chemotherapeutic agent.

In some embodiments, administering the effective amount of Eflapegrastim may reduce the duration of an absolute neutrophil count of less than about 0.5×10⁹/L in the patient to less than about 24 hours. Specifically, administering the effective amount of Eflapegrastim may reduce the duration of an absolute neutrophil count of less than about 0.5×10⁹/L in the patient to less than about 24 hours, about 12 hours, or about 8 hours. More specifically, administering the effective amount of Eflapegrastim may reduce the duration of an absolute neutrophil count of less than about 0.5×10⁹/L in the patient to about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In an embodiment, administering the effective amount of Eflapegrastim reduces the duration of an absolute neutrophil count of less than about 0.5×10⁹/L in the patient to less than about 24 hours. In an embodiment, administering the effective amount of Eflapegrastim reduces the duration of an absolute neutrophil count of less than about 0.5×10⁹/L in the patient to less than about 12 hours. In an embodiment, administering the effective amount of Eflapegrastim reduces the duration of an absolute neutrophil count of less than about 0.5×10⁹/L in the patient to less than about 8 hours.

In some embodiments, administering the effective amount of Eflapegrastim prevents the absolute neutrophil count in the patient from reaching less than about 0.5×10⁹/L.

In some embodiments, the chemotherapy-induced neutropenia is severe neutropenia with an absolute neutrophil count less than 0.5×10⁹/L and upon administration of the effective amount of Eflapegrastim, an absolute neutrophil count of the patient increases from the first occurrence of less than about 0.5×10⁹/L to greater than or equal to about 1.5×10⁹/L within less than about four days, about seven days, or about ten days. Specifically, the time for recovery of absolute neutrophil count in the patient from the first occurrence of less than about 0.5×10⁹/L to an absolute neutrophil count of greater than or equal to about 1.5×10⁹/L is less than about ten days, about seven days, or about four days. In certain embodiments, the chemotherapy-induced neutropenia is severe neutropenia with an absolute neutrophil count less than 0.5×10⁹/L and the time for recovery from an absolute neutrophil count of less than about 0.5×10⁹/L in the patient to an absolute neutrophil count of greater than or equal to about 1.5×10⁹/L in the patient is less than about one day, about two days, about three days, about four days, about five days, about six days, about seven days, about eight days, about nine days, or about ten days

In some embodiments, the method is for increasing the absolute neutrophil count in a patient in need thereof and the time for recovery from an absolute neutrophil count of less than about 0.5×10⁹/L in the patient to an absolute neutrophil count of greater than or equal to about 1.5×10⁹/L in the patient is less than about ten days. Specifically, the time for recovery of absolute neutrophil count of less than about 0.5×10⁹/L in the patient to an absolute neutrophil count of greater than or equal to about 1.5×10⁹/L is less than about ten days, about seven days, or about four days. In certain embodiments, the time for recovery from an absolute neutrophil count of less than about 0.5×10⁹/L in the patient to an absolute neutrophil count of greater than or equal to about 1.5×10⁹/L in the patient is less than about one day, about two days, about three days, about four days, about five days, about six days, about seven days, about eight days, about nine days, or about ten days.

In an embodiment, the effective amount of Eflapegrastim is administered concomitantly with the chemotherapeutic agent.

In certain embodiments, the effective amount of Eflapegrastim is administered within about 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, after the administration of the chemotherapeutic agent.

In certain embodiments, the effective amount of Eflapegrastim is administered within about 0.5 hours, about 1 hour, about 2 hours, about 3 hours, about 4 hours, about 5 hours, about 6 hours, about 7 hours, about 8 hours, about 9 hours, about 10 hours, about 11 hours, or about 12 hours after the administration of the chemotherapeutic agent.

In certain embodiments, the effective amount of Eflapegrastim is administered within about 0.5 hours, about 3 hours, or about 5 hours after the administration of the chemotherapeutic agent.

In an embodiment, the chemotherapeutic agent is a myelosuppressive chemotherapeutic agent.

In certain embodiments, the myelosuppressive chemotherapeutic agent is selected from the group consisting of docetaxel, cyclophosphamide, doxorubicin, etoposide, cisplatin, paclitaxel, topotecan, vincristine, methylprednisolone, cytarabine, and combinations thereof.

In certain embodiments, the patient is receiving the chemotherapeutic agent to treat a cancer selected from the group consisting of breast cancer, non-small cell lung cancer, small cell lung cancer, ovarian cancer, sarcoma, urothelial cancer, germ cell tumors and non-Hodgkin's lymphoma.

In some embodiments, administering an effective amount of Eflapegrastim comprises administering parenterally to a patient at a dosage from about 2 to 18 mg of Eflapegrastim. In an embodiment, the dosage may be about 13.2 mg of Eflapegrastim per day.

In certain embodiments, administering an effective amount of Eflapegrastim comprises administering parenterally at a dosage from about 2.0 to about 5.0 mg, about 5.0 mg to about 15.0 mg, about 7.0 mg to about 15.0 mg, about 9.0 mg to about 15.0 mg, about 11.0 mg to about 15.0 mg, about 13.0 mg to about 15.0 mg, about 5.0 mg to about 13.0 mg, about 5.0 mg to about 11.0 mg, about 5.0 mg to about 9.0 mg, about 5.0 mg to about 7.0 mg, about 7.0 mg to about 13.0 mg, about 7.0 mg to about 11.0 mg, about 7.0 mg to about 9.0 mg, about 9.0 mg to about 13.0 mg, about 9.0 mg to about 11.0 mg, about 11.0 mg to about 13.0 mg, or about 15.0 to about 18.0 mg of Eflapegrastim.

In certain embodiments, administering an effective amount of Eflapegrastim comprises administering parenterally about 12.0 mg, about 12.2 mg, about 12.4 mg, about 12.6 mg, about 12.8 mg, about 13.0 mg, about 13.2 mg, about 13.4 mg, about 13.6 mg, about 13.8 mg, or about 14.0 mg of Eflapegrastim. In certain embodiments, administering an effective amount of Eflapegrastim comprises administering parenterally about 13.2 mg of Eflapegrastim.

Specifically, the dosage of Eflapegrastim may be administered as a single dose, or may be divided into 1 to 5 doses, within 24 hours from the administration of a chemotherapeutic agent, optionally on the same day when the patient is administered the chemotherapeutic agent.

### Treatment of Radiotherapy-Induced Neutropenia

In one aspect, provided herein is a method for increasing the absolute neutrophil count, the number of granulocytes in a subject eligible for a bone marrow transplant, stem cell production, hematopoiesis, the number of hematopoietic progenitor cells, or stem cell production in a donor, comprising administering an effective amount of Eflapegrastim within a period of less than 24 hours after the patient receives a radiotherapy.

In another aspect, provided herein is a method for treating or preventing the condition characterized by compromised white blood cell production in a patient in need thereof, comprising administering an effective amount of Eflapegrastim within a period of less than 24 hours after the patient receives a radiotherapy.

In some embodiments, the condition characterized by compromised white blood cell production is selected from the group consisting of: radiotherapy-induced neutropenia, reduced hematopoietic function, reduced immune function, reduced neutrophil count, reduced neutrophil mobilization, mobilization of peripheral blood progenitor cells, sepsis, bone marrow transplants, infectious diseases, leucopenia, thrombocytopenia, anemia, enhancing engraftment of bone marrow during transplantation, enhancing bone marrow recovery in treatment of radiation, radiotherapy induced bone marrow aplasia or myelosuppression, and acquired immune deficiency syndrome

In an embodiment, the condition is a radiotherapy-induced neutropenia. In an embodiment, the method may reduce the duration of radiotherapy-induced neutropenia in a patient in need thereof.

In an embodiment, the method comprises administering an effective amount of Eflapegrastim on the same day when the patient receives radiotherapy.

In certain embodiments, administering the effective amount of Eflapegrastim may reduce the duration of an absolute neutrophil count of less than about 0.5×10⁹/L in the patient to less than about 24 hours. Specifically, administering the effective amount of Eflapegrastim may reduce the duration of an absolute neutrophil count of less than about 0.5×10⁹/L in the patient to less than about 24 hours, about 12 hours, or about 8 hours. More specifically, administering the effective amount of Eflapegrastim may reduce the duration of an absolute neutrophil count of less than about 0.5×10⁹/L in the patient to about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In an embodiment, administering the effective amount of Eflapegrastim reduces the duration of an absolute neutrophil count of less than about 0.5×10⁹/L in the patient to less than about 24 hours. In an embodiment, administering the effective amount of Eflapegrastim reduces the duration of an absolute neutrophil count of less than about 0.5×10⁹/L in the patient to less than about 12 hours. In an embodiment, administering the effective amount of Eflapegrastim reduces the duration of an absolute neutrophil count of less than about 0.5×10⁹/L in the patient to less than about 8 hours.

In some embodiments, administering the effective amount of Eflapegrastim prevents the absolute neutrophil count in the patient from reaching less than about 0.5×10⁹/L. In some embodiments, the radiotherapy-induced neutropenia is severe neutropenia with an absolute neutrophil count less than 0.5×10⁹/L and upon administration of the effective amount of Eflapegrastim, an absolute neutrophil count of the patient increases from the first occurrence of less than about 0.5×10⁹/L to greater than or equal to about 1.5×10⁹/L within less than about four days, about seven days, or about ten days. Specifically, the time for recovery of absolute neutrophil count in the patient from the first occurrence of less than about 0.5×10⁹/L to an absolute neutrophil count of greater than or equal to about 1.5×10⁹/L is less than about ten days, about seven days, or about four days. In certain embodiments, the radiotherapy-induced neutropenia is severe neutropenia with an absolute neutrophil count less than 0.5×10⁹/L and the time for recovery from an absolute neutrophil count of less than about 0.5×10⁹/L in the patient to an absolute neutrophil count of greater than or equal to about 1.5×10⁹/L in the patient is less than about one day, about two days, about three days, about four days, about five days, about six days, about seven days, about eight days, about nine days, or about ten days

In some embodiments, the method is for increasing the absolute neutrophil count in a patient in need thereof and the time for recovery from an absolute neutrophil count of less than about 0.5×10⁹/L in the patient to an absolute neutrophil count of greater than or equal to about 1.5×10⁹/L in the patient is less than about ten days. Specifically, the time for recovery of absolute neutrophil count of less than about 0.5×10⁹/L in the patient to an absolute neutrophil count of greater than or equal to about 1.5×10⁹/L is less than about ten days, about seven days, or about four days.

In certain embodiments, the time for recovery from an absolute neutrophil count of less than about 0.5×10⁹/L in the patient to an absolute neutrophil count of greater than or equal to about 1.5×10⁹/L in the patient is less than about one day, about two days, about three days, about four days, about five days, about six days, about seven days, about eight days, about nine days, or about ten days.

In an embodiment, the effective amount of Eflapegrastim is administered concomitantly with the receipt of the radiotherapy.

In certain embodiments, the effective amount of Eflapegrastim is administered within about 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, after the receipt of the radiotherapy.

In certain embodiments, the effective amount of Eflapegrastim is administered within about 0.5 hours, about 1 hour, about 2 hours, about 3 hours, about 4 hours, about 5 hours, about 6 hours, about 7 hours, about 8 hours, about 9 hours, about 10 hours, about 11 hours, or about 12 hours after the receipt of the radiotherapy.

In certain embodiments, the effective amount of Eflapegrastim is administered within about 0.5 hours, about 3 hours, or about 5 hours after the receipt of the radiotherapy.

In certain embodiments, the patient is receiving the radiotherapy to treat a cancer selected from the group consisting of breast cancer, non-small cell lung cancer, small cell lung cancer, ovarian cancer, sarcoma, urothelial cancer, germ cell tumors and non-Hodgkin's lymphoma.

In some embodiments, administering an effective amount of Eflapegrastim comprises administering parenterally to a patient at a dosage from about 2 to 18 mg of Eflapegrastim. In an embodiment, the dosage may be about 13.2 mg of Eflapegrastim per day.

In certain embodiments, administering an effective amount of Eflapegrastim comprises administering parenterally at a dosage from about at a dosage from about 2.0 to about 5.0 mg, about 5.0 mg to about 15.0 mg, about 7.0 mg to about 15.0 mg, about 9.0 mg to about 15.0 mg, about 11.0 mg to about 15.0 mg, about 13.0 mg to about 15.0 mg, about 5.0 mg to about 13.0 mg, about 5.0 mg to about 11.0 mg, about 5.0 mg to about 9.0 mg, about 5.0 mg to about 7.0 mg, about 7.0 mg to about 13.0 mg, about 7.0 mg to about 11.0 mg, about 7.0 mg to about 9.0 mg, about 9.0 mg to about 13.0 mg, about 9.0 mg to about 11.0 mg, about 11.0 mg to about 13.0 mg, or about 15.0 to about 18.0 mg of Eflapegrastim.

In certain embodiments, administering an effective amount of Eflapegrastim comprises administering parenterally about 12.0 mg, about 12.2 mg, about 12.4 mg, about 12.6 mg, about 12.8 mg, about 13.0 mg, about 13.2 mg, about 13.4 mg, about 13.6 mg, about 13.8 mg, or about 14.0 mg of Eflapegrastim. In certain embodiments, administering an effective amount of Eflapegrastim comprises administering parenterally about 13.2 mg of Eflapegrastim.

Specifically, the dosage of Eflapegrastim may be administered as a single dose, or may be divided into 1 to 5 doses, within 24 hours from the receipt of radiotherapy, optionally on the same day when the patient receives the radiotherapy.

### EXAMPLES

In order that the disclosure described herein is more fully understood, the following examples are set forth. The synthetic and biological examples described in this application are offered to illustrate the compounds, pharmaceutical compositions, and methods provided herein and are not to be construed in any way as limiting their scope.

### Example 1: Preparation of Eflapegrastim (^{17,65}S-G-CSF-PEG-Fc)

### Step 1: Preparation of Immunoglobulin Fc Fragment Using Immunoglobulin

Preparation of an immunoglobulin Fc fragment was prepared as follows.

200 mg of 150-kDa immunoglobulin G (IgG) (Green Cross, Korea) dissolved in 10 mM phosphate buffer was treated with 2 mg of a proteolytic enzyme, papain (Sigma) at 37° C. for 2 hrs with gentle agitation.

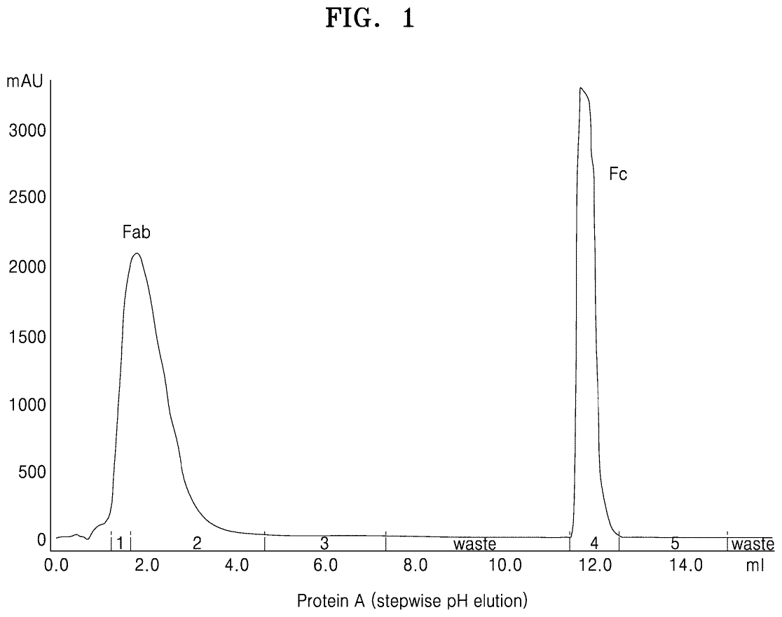

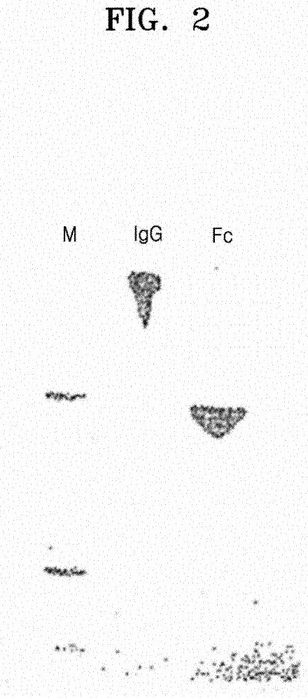

After the enzyme reaction, the immunoglobulin Fc fragment regenerated thus was subjected to chromatography for purification using sequentially a Superdex column, a protein A column and a cation exchange column. In detail, the reaction solution was loaded onto a Superdex 200 column (Pharmacia) equilibrated with 10 mM sodium phosphate buffer (PBS, pH 7.3), and the column was eluted with the same buffer at a flow rate of 1 ml/min. Unreacted immunoglobulin molecules (IgG) and F(ab′)2, which had a relatively high molecular weight compared to the immunoglobulin Fc fragment, were removed using their property of being eluted earlier than the Ig Fc fragment. Fab fragments having a molecular weight similar to the Ig Fc fragment were eliminated by protein A column chromatography (FIG. 1). The resulting fractions containing the Ig Fc fragment eluted from the Superdex 200 column were loaded at a flow rate of 5 ml/min onto a protein A column (Pharmacia) equilibrated with 20 mM phosphate buffer (pH 7.0), and the column was washed with the same buffer to remove proteins unbound to the column. Then, the protein A column was eluted with 100 mM sodium citrate buffer (pH 3.0) to obtain highly pure immunoglobulin Fc fragment. The Fc fractions collected from the protein A column were finally purified using a cation exchange column (polyCAT, PolyLC Company), wherein this column loaded with the Fc fractions was eluted with a linear gradient of 0.15-0.4 M NaCl in 10 mM acetate buffer (pH 4.5), thus providing highly pure Fc fractions. The highly pure Fc fractions were analyzed by 12% SDS-PAGE (lane 2 in FIG. 2).

### Step 2: Preparation of ^{17,65}S-G-CSF-PEG Complex

3.4-kDa polyethylene glycol having an aldehyde reactive group at both ends, ALD-PEG-ALD (Shearwater), was mixed with human granulocyte colony stimulating factor (^{17,65}S-G-CSF, MW: 18.6 kDa) dissolved in 100 mM phosphate buffer in an amount of 5 mg/ml at a ^{17,65}S-G-CSF:PEG molar ratio of 1:5. To this mixture, a reducing agent, sodium cyanoborohydride (NaCNBH₃, Sigma), was added at a final concentration of 20 mM and was allowed to react at 4° C. for 3 hrs with gentle agitation to allow PEG to link to the amino terminal end of ^{17,65}S-G-CSF. To obtain a 1:1 complex of PEG and ^{17,65}S-G-CSF, the reaction mixture was subjected to size exclusion chromatography using a SUPERDEX^{R} column (Pharmacia). The ^{17,65}S-G-CSF-PEG complex was eluted from the column using 10 mM potassium phosphate buffer (pH 6.0) as an elution buffer, and ^{17,65}S-G-CSF not linked to PEG, unreacted PEG and dimer byproducts where PEG was linked to ^{17,65}S-G-CSF molecules were removed. The purified ^{17,65}S-G-CSF-PEG complex was concentrated to 5 mg/ml. Through this experiment, the optimal reaction molar ratio for ^{17,65}S-G-CSF to PEG, providing the highest reactivity and generating the smallest amount of byproducts such as dimers, was found to be 1:5.

### Step 3: Preparation of the ^{17,65}S-G-CSF-PEG-Fc Conjugate

To link the ^{17,65}S-G-CSF-PEG complex purified in the above step 2 to the terminus of an immunoglobulin Fe fragment, the immunoglobulin Fe fragment (about 53 kDa) prepared in Step 1 was dissolved in 10 mM phosphate buffer and mixed with the ^{17,65}S-G-CSF-PEG complex at an ^{17,65}S-G-CSF-PEG complex:Fc molar ratio of 1:1, 1:2, 1:4 and 1:8. After the phosphate buffer concentration of the reaction solution was adjusted to 100 mkt, a reducing agent, NaCNBH₃, was added to the reaction solution at a final concentration of 20 mM and was allowed to react at 4° C. for 20 hrs with gentle agitation. Through this experiment, the optimal reaction molar ratio for ^{17,65}S-G-CSF-PEG complex to Fc, providing the highest reactivity and generating the fewest byproducts such as dimers, was found to be 1:2.

### Step 4: Isolation and Purification of the G-CSF-PEG-Fc Conjugate

After the reaction of the above step 3, the reaction mixture was subjected to Superdex size exclusion chromatography so as to eliminate unreacted substances and byproducts and purify the ^{17,65}S-G-CSF-PEG-Fc protein conjugate produced. After the reaction mixture was concentrated and loaded onto a Superdex column, 10 mM phosphate buffer (pH 7.3) was passed through the column at a flow rate of 2.5 ml/min to remove unbound Fc and unreacted substances, followed by column elution to collect ^{17,65}S-G-CSF-PEG-Fc protein conjugate fractions. Since the collected ^{17,65}S-G-CSF-PEG-Fc protein conjugate fractions contained a small amount of impurities, unreacted Fc and interferon alpha dimers, cation exchange chromatography was carried out to remove the impurities. The ^{17,65}S-G-CSF-PEG-Fc protein conjugate fractions were loaded onto a Polyp AT LP column (PolyLC) equilibrated with 10 mM sodium acetate (pH 4.5), and the column was eluted with a linear gradient of 0-0.5 M NaCl in 10 mM sodium acetate buffer (pH 4.5) using 1 M NaCl. Finally, the ^{17,65}S-G-CSF-PEG-Fc protein conjugate was purified using an anion exchange column. The ^{17,65}S-G-CSF-PEG-Fc protein conjugate fractions were loaded onto a PolyWAX LP column (PolyLC) equilibrated with 10 mM Tris-HCl (pH 7.5), and the column was then eluted with a linear gradient of 0-0.3 M NaCl in 10 mM Tris-HCl (pH 7.5) using 1 M NaCl, thus isolating the ^{17,65}S-G-CSF-PEG-Fc protein conjugate in a highly pure form.

### Example 2: Efficacy Study of Eflapegrastim by Different Dosing Regimens in Rats with Docetaxel/Cyclophosphamide Induced Neutropenia

The efficacy of Eflapegrastim (HM10460A), a long acting G-CSF analogue, was compared with Pegfilgrastim by different dosing regimens in a chemotherapy-induced neutropenic rat model.

In the following study, the Eflapegrastim was created essentially as described in Example 1.

### (i) Materials for Study

TABLE 1
Test Articles

| | | | | Expira- | |
|---|---|---|---|---|---|
| | Batch/Lot | Storage | Purity | tion | |
| Name | No. | Condition | (%) | Date | Supplier |
| HM10460A | 906617001 | 2~8° C. | RP-HPLC: | Jan. 31, | — |
| | | | 98.6% | 2019 | |
| | | | IE-HPLC: | | |
| | | | 97.4% | | |
| | | | SE-HPLC: | | |
| | | | 98.6% | | |
| Pegfilgrastim | 1070334 | 2~8° C. | — | — | Amgen |

TABLE 2
Vehicles

| | | Storage | |
|---|---|---|---|
| Name | Composition | Condition | Supplier |
| Dulbecco's phosphate | — | 2~8° C. | Sigma-Aldrich |
| buffered saline (DPBS) | | | |

TABLE 3
Neutropenia-Inducing Agents

| | Batch/Lot | Storage | Purity | Expiration | |
|---|---|---|---|---|---|
| Name | No. | Condition | (%) | Date | Supplier |
| Cyclophosphamide | C3250000 | 2~8° C. | — | — | Sigma-Aldrich |
| Docetaxel | 17006 | RT (20-25° C.) | — | Oct. 31, | Hanmi |
| | | | | 2020 | Pharmaceutical Co. |

### Preparing HM10460A Solutions for Subcutaneous Administration

Preparation of a 61.8 μg/kg HM10460A solution for subcutaneous administration: a stock solution of HM10460A (6.0 mg/mL) 92.7 μL was diluted with DPBS 17907.3 μL.

Preparation of a 372.0 μg/kg HM10460A solution for subcutaneous administration: a stock solution of HM10460A (6.0 mg/mL) 558.0 μL was diluted with DPBS 17442.0 μL.

Preparation of a 496.0m/kg HM10460A solution for subcutaneous administration: a stock solution of HM10460A (6.0 mg/mL) 744.0 μL was diluted with DPBS 17256.0 μL.

The test article was prepared based on G-CSF protein dosage on drug label (HM10460A.)

The HM10460A solution for subcutaneous administration was then diluted with DPBS to a final dose concentration of 2 mL/kg.

### Preparing Pegfilgrastim Solutions for Subcutaneous Administration

Preparation of a 103.3 μg/kg Pegfilgrastim solution for subcutaneous administration: a stock solution of Pegfilgrastim (10 mg/mL) 93.0 μL was diluted with DPBS 17907.0 μL.

Preparation of a 620.0m/kg Pegfilgrastim solution for subcutaneous administration: a stock solution of Pegfilgrastim (10 mg/mL) 558.0 μL was diluted with DPBS 17442.0 μL.

The Pegfilgrastim solution for subcutaneous administration was then diluted with DPBS to a final dose concentration of 2 mL/kg.

### Preparing Solutions of Neutropenia-Inducing Agents

To induce neutropenia in rats, Docetaxel/cyclophosphamide was administered using a ⅓ human equivalent dose (Docetaxel 4 mg/kg and CPA 32 mg/kg) (“TC”).

Preparation of a 32 mg/kg cyclophosphamide solution for subcutaneous administration: cyclophosphamide powder (CPA, Sigma, USA) 2560.0 g was diluted with distilled water (DW, Daihan, Korea) 80000.0 μL.

Preparation of a 4 mg/kg docetaxel solution for subcutaneous administration: Docel inj. (Hanmi Pharmaceutical, Korea) (42.68 mg/mL) 29070.0 μL was diluted with a commercial formulation buffer (FB, Ethanol 127.4 mg/mL in DW) 30930.0 μL.

The docetaxel and cyclophosphamide solutions for subcutaneous administration were then diluted with FB to a final dose concentration of 1 mL/kg. HM10460A and Pegfilgrastim were diluted with DPBS to a final dose concentration of 2 mL/kg.

### (ii) Methods

### Test System

TABLE 4

| | |
|---|---|
| Species and | Rats |
| Strain | Crl: CD Sprague Dawley (SD) |
| Justification | SD rats were chosen due to their extensive |
| for Species | characterization collected from various |
| | preclinical studies, especially with the |
| | study done to test G-CSF analogue1), 2). |
| Supplier | Orient Bio corp. Korea |
| | 143-1, Sangdaewondong, Jungwon-gu, Seongnam-si, |
| | Gyeonggi-do, Korea |
| Number of | Male 125 (at group allocation) |
| animals | |
| Age | 8 weeks (at group allocation) |
| Body weight | 239.54~316.46 g (at start of dosing) |
| range | |
| Neutropenia | Normal SD rats were administered with Docetaxel |
| induction | 4 mg/kg and CPA 32 mg/kg once intraperitoneally |
| with | to induce neutropenia. Docetaxel and CPA were |
| chemotherapy | injected to induce neutropenia in a rat model |
| | according to 4 different regimens: Concomitant |
| | (G2-G7), 2 hour (G8-G13), 5 hour (G14-G19), |
| | and 24 hour (G20-G25) prior to test article |
| | administration. |

### Animal Care and Identification

TABLE 5

| | |
|---|---|
| Acclimation | 7 days before commencement of treatment |
| Disposition | Extra animals were sacrificed at the beginning of the |
| of extra and | study using CO₂ gas. Also experimental animals were |
| experimental | euthanized using CO₂ gas at final measurement. |
| animals | |
| Group | Five rats were assigned in each group according to the |
| assignment | ANC profile. |
| Identification | Cage card and tail mark |

### Animal Husbandry

TABLE 6

| | |
|---|---|
| Housing | Clean barrier |
| Cage | Polysulfone cage 1291H (W425 × D266 × H185 mm, |
| | Tecniplast, Italy). |
| No. of Animal | 3 rats per cage |
| Environment | Tempeature: 22 ± 2° C. |
| | Relative Humidity: 50 ± 20% |
| | Ventilation frequency: 10-15 times/hour |
| | Light/dark cycle: 12 hour (AM 6:00-PM 6:00) |
| | Light intensity: 150-300 Lux |
| | Frequency of replacement of the cage: At least once |
| | weekly |
| Diet | The pellet chows (PICOLAB ® Rodent Diet 20 (5053, |
| | LabDiet, USA)) were given ad libitum. |
| Drinking Water | The tap water was given ad libitum, following the |
| | filtration. |
| Monitoring the | Throughout the study, the temperature and relative |
| housing | humidity of the animal room was automatically |
| conditions | controlled and recorded at every 30 minutes. The |
| | light intensity was periodically monitored. |

### Dose Administration

TABLE 7

| | |
|---|---|
| Administration | HM10460A: 61.8 μg/kg, 372.0 μg/kg and 496.0 μg/kg |
| volume | Pegfilgrastim: 103.3 μg/kg and 620.0 μg/kg |
| Duration of | Once |
| treatment | |
| Dosage | HM10460A and Pegfilgrastim were administrated at the |
| | clinical dose (372.0 and 620.0 μg/kg, respectively) or 1/6 |
| | clinical dose (61.8 and 103.3 μg/kg, respectively) |
| | considering body surface area of rats). Additional |
| | testing was performed using a test article including, |
| | a higher dose of HM10460A, 496.0 μg/kg. |
| Fasting | Animals were not fasted. |
| Administration | Animals were administered either through subcutaneous |
| route | route to dorsal site (s.c., test articles) or |
| | intraperitoneal route to abdominal site (i.p., |
| | Docetaxel and Cyclophosphamide). |
| Volume of | 2 mL/kg (test articles) and 1 mL/kg (Docetaxel and |
| administration | Cyclophosphamide) |

### Group Design and Dose Level

TABLE 8

| | | | Dose | Human | | | | |
|---|---|---|---|---|---|---|---|---|
| | | | (μg/kg | dose | | | | |
| | TC | | as G- | (mg/head as | | | No. of | Individual |
| Group | admin | Test Article | CSF) | G-CSF)* | Route | Frequency | animals | No. |
| G1 | — | Normal | — | — | s.c. | Once | 5 | M01- |
| | | | | | | | | M05 |
| G2 | D0 | †TC, vehicle | — | — | s.c. | Once | 5 | M06- |
| | + 0 h | | | | | | | M10 |
| G3 | | †TC, | 103.3 | 1.0 | s.c. | Once | 5 | M11- |
| | | Pegfilgrastim | | | | | | M15 |
| G4 | | | 620.0 | 6.0 | s.c. | Once | 5 | M16- |
| | | | | | | | | M20 |
| G5 | | †TC, | 61.8 | 0.6 | s.c. | Once | 5 | M21- |
| | | HM10460A | | | | | | M25 |
| G6 | | | 372.0 | 3.6 | s.c. | Once | 5 | M26- |
| | | | | | | | | M30 |
| G7 | | | 496.0 | 4.8 | s.c. | Once | 5 | M31- |
| | | | | | | | | M35 |
| G8 | D0 | †TC, vehicle | — | — | s.c. | Once | 5 | M36- |
| | +2 h | | | | | | | M40 |
| G9 | | †TC, | 103.3 | 1.0 | s.c. | Once | 5 | M41- |
| | | Pegfilgrastim | | | | | | M45 |
| G10 | | | 620.0 | 6.0 | s.c. | Once | 5 | M46- |
| | | | | | | | | M50 |
| G11 | | †TC, | 61.8 | 0.6 | s.c. | Once | 5 | M51- |
| | | HM10460A | | | | | | M55 |
| G12 | | | 372.0 | 3.6 | s.c. | Once | 5 | M56- |
| | | | | | | | | M60 |
| G13 | | | 496.0 | 4.8 | s.c. | Once | 5 | M61- |
| | | | | | | | | M65 |
| G14 | D0 | †TC, vehicle | — | — | s.c. | Once | 5 | M66- |
| | +5 h | | | | | | | M70 |
| G15 | | †TC, | 103.3 | 1.0 | s.c. | Once | 5 | M71- |
| | | Pegfilgrastim | | | | | | M75 |
| G16 | | | 620.0 | 6.0 | s.c. | Once | 5 | M76- |
| | | | | | | | | M80 |
| G17 | | †TC, | 61.8 | 0.6 | s.c. | Once | 5 | M81- |
| | | HM10460A | | | | | | M85 |
| G18 | | | 372.0 | 3.6 | s.c. | Once | 5 | M86- |
| | | | | | | | | M90 |
| G19 | | | 496.0 | 4.8 | s.c. | Once | 5 | M91- |
| | | | | | | | | M95 |
| G20 | D0 | †TC, vehicle | — | — | s.c. | Once | 5 | M96- |
| | +24 h | | | | | | | M100 |
| G21 | | †TC, | 103.3 | 1.0 | s.c. | Once | 5 | M101- |
| | | Pegfilgrastim | | | | | | M105 |
| G22 | | | 620.0 | 6.0 | s.c. | Once | 5 | M106- |
| | | | | | | | | M110 |
| G23 | | †TC, | 61.8 | 0.6 | s.c. | Once | 5 | M111- |
| | | HM10460A | | | | | | M115 |
| G24 | | | 372.0 | 3.6 | s.c. | Once | 5 | M116- |
| | | | | | | | | M120 |
| G25 | | | 496.0 | 4.8 | s.c. | Once | 5 | M121- |
| | | | | | | | | M125 |

†Docetaxel and CPA were injected to induce neutropenia in the rats according to 4 different regimens: Concomitant (G2-G7), 2 hour .(G8-G13), 5 hour (G14-G19), and 24 hour (G20-G25) prior to test article administration.
*Corresponding human dose. Reagan-Shaw, Nihal M., Almad N., Dose Translation from animal to human studies revisited, FASEB J. 2008 March; 22(3): 659-61.

(iii) Observations and Measurements

### Body Weight

Body weight was measured twice at day −1 and day 0 once prior to TC and test article dosing to calculate for proper volume administration.

### ANC Profile

All animal blood was collected from the jugular vein on the day −1 before chemotherapy and analyzed for neutrophil count (NEUT #). This neutrophil count was used as NEUT of day 0 before dosing and groupings were based on NEUT of day 0. Also, blood was collected at 6 hrs in day 0 and once a day for 8 days after test article administration with a 26G 1 mL syringe. 0.2 mL total blood was collected and put into automatic blood corpuscle analyzer Sysmex, XN1000-V (Sysmex corp., Japan) to check ANC. Though ANC is normally calculated from total WBC×(% Segs+% Bands), ANC can be calculated using the Sysmex system because the quantity of neutrophils measured with the Sysmex system already includes neutrophil band type in the data.

### Duration of Neutropenia Profile

The primary end point for this study was determined from the duration of neutropenia (“DN”), which was determined based on the cut off values on neutrophil level calculated from normal vehicle (mean of overall neutrophil level).

### (iv) Results

### ANC Profile

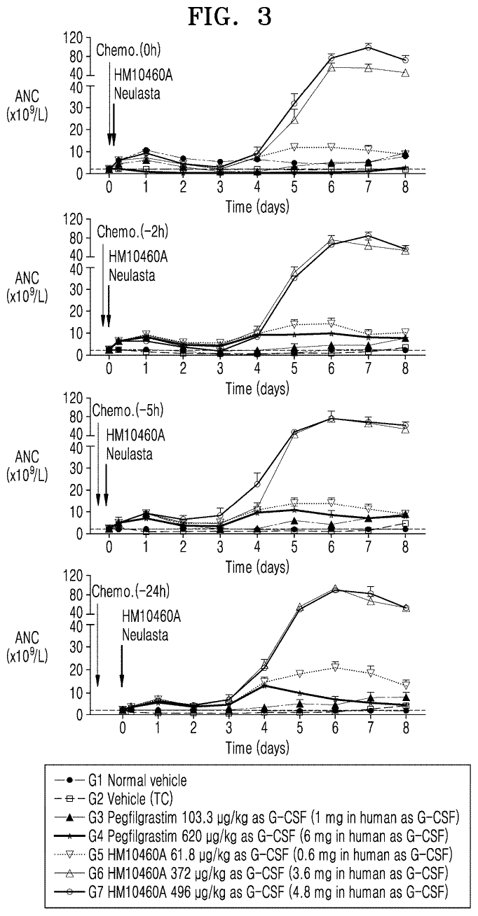

The time course of the neutrophil count is shown in FIG. 3. The neutrophil count at ⅙ clinical dose (Pegfilgrastim 103.3 μg/kg and HM10460A 61.8 μg/kg) reached its peak on the day 8 and day 5-6 after the start of drug administration for Pegfilgrastim and HM10460A without any difference between dosing regimen, respectively. Also, the neutrophil count at clinical dose (Pegfilgrastim 620 μg/kg and HM10460A 372 μg/kg) reached its peak on the day 5-8 and day 6 after the start of drug administration for Pegfilgrastim and HM10460A, respectively. Moreover, the peak of the neutrophil count was between days 6 and 7 for HM10460A high dose (496 μg/kg) in all time regimen with no dosing regimen changes.

### DN Profile

At ⅙ clinical dose (HM10460A 61.8 μg/kg and Pegfilgrastim 103.3 μg/kg), the DN value of HM10460A and Pegfilgrastim administered 24 hours after chemotherapy was determined to be 0.2 and 1.8 days, respectively (TABLE 9). As the interval between the chemotherapy and the test article being administered became shorter (5 hours, 2 hours, and concomitant), the DN of Pegfilgrastim increased to 2.4 days. By comparison, only a slight increase to 0.6 days was observed for HM10460A.

When administering the clinical dose (HM10460A 372 μg/kg and Pegfilgrastim 620 μg/kg), the DN of HM10460A and Pegfilgrastim administered at 24 hours after chemotherapy was observed to be 0 and 0.2 days, respectively (TABLE 10). As the interval between the chemotherapy and the test article being administered became shorter (5 hours, 2 hours, and concomitant), the DN of Pegfilgrastim was increased to 1.4 days. The DN as a result of administration of HM10460A, on the other hand, increased only slightly to 0.6 days, as was observed for the ⅙ clinical dose.

The high dose of HM10460A (496 μg/kg) showed similar profile (0.2 day) regardless of time of administration, except for the D0+2 h regimen (TABLE 11).

TABLE 9
Comparison of DN Values (1/6 Clinical Dose)

| 1/6 clinical dose | | | | | | | | | |
|---|---|---|---|---|---|---|---|---|---|
| Pegfilgrastim: | | Concomitant or Within-a-day | | | | | | Sequential | |
| 103.3 μg/kg | | +0 hr | | +2 hr | | +5 hr | | +24 hr | |
| HM10460A: | | Peg- | | Peg- | | Peg- | | Peg- | |
| 61.8 μg/kg | | filgrastim | HM10460A | filgrastim | HM10460A | filgrastim | HM10460A | filgrastim | HM10460A |
| DN (day) | 0 | 0 | 2 | 1 | 4 | 1 | 0 | 1 | 4 |
| Vehicle: | 1 | 1 | 3 | 1 | 1 | 2 | 0 | 1 | 1 |
| Con 7.2 | 2 | 2 | 0 | 2 | 0 | 2 | 0 | 1 | 0 |
| +2 h 7.0 | 3 | 2 | 0 | 0 | 0 | 0 | 0 | 2 | 0 |
| +5 h 7.4 | 4 | 0 | 0 | 1 | 0 | 0 | 0 | 0 | 0 |
| Seq 7.0 | | | | | | | | | |

| | | | | | | | | |
|---|---|---|---|---|---|---|---|---|
| Average DN | 2.2 | 0.6 | 1.8 | 0.2 | 1.2 | 0.0 | 1.8 | 0.2 |
| (day) | | | | | | | | |

*‘+’ stands for the interval from the administration of the chemotherapy to the administration of the test articles.

TABLE 10
Comparison of DN Values (Clinical Dose)

| Clinical dose | | | | | | | | | |
|---|---|---|---|---|---|---|---|---|---|
| Pegfilgrastim: | | Concomitant or Within-a-day | | | | | | Sequential | |
| 620 μg/kg | | +0 hr | | +2 hr | | +5 hr | | +24 hr | |
| HM10460A: | | Peg- | | Peg- | | Peg- | | Peg- | |
| 372 μg/kg | | filgrastim | HM10460A | filgrastim | HM10460A | filgrastim | HM10460A | filgrastim | HM10460A |
| DN (day) | 0 | 3 | 2 | 1 | 4 | 3 | 4 | 4 | 5 |
| Vehicle: | 1 | 0 | 3 | 2 | 0 | 1 | 1 | 1 | 0 |
| Con 7.2 | 2 | 1 | 0 | 1 | 1 | 1 | 0 | 0 | 0 |
| +2 h 7.0 | 3 | 1 | 0 | 1 | 0 | 0 | 0 | 0 | 0 |
| +5 h 7.4 | 4 | 0 | 0 | 0 | 0 | 0 | 0 | 0 | 0 |
| Seq 7.0 | | | | | | | | | |

| | | | | | | | | |
|---|---|---|---|---|---|---|---|---|
| Average DN | 1.0 | 0.6 | 1.4 | 0.4 | 0.6 | 0.2 | 0.2 | 0 |
| (day) | | | | | | | | |

*‘+’ stands for the interval from the administration of the chemotherapy to the administration of the test articles.

TABLE 11
Comparison of DN Values (Clinical Dose)

| High dose HM10460A- | | Concomitant or Within-a-day | | | Sequential |
|---|---|---|---|---|---|
| HM10460A: | | +0 hr | +2 hr | +5 hr | +24 hr |
| 496 μg/kg | | HM10460A | HM10460A | HM10460A | HM10460A |
| DN (day) | 0 | 4 | 1 | 4 | 4 |
| Vehicle: | 1 | 1 | 3 | 1 | 1 |
| Con 7.2 | 2 | 0 | 1 | 0 | 0 |
| +2 h 7.0 | 3 | 0 | 0 | 0 | 0 |
| +5 h 7.4 | 4 | 0 | 0 | 0 | 0 |
| Seq 7.0 | | | | | |

| | | | | |
|---|---|---|---|---|
| Average DN (day) | 0.2 | 1.0 | 0.2 | 0.2 |

*+stands for the interval from the administration of the chemotherapy to the administration of the test articles.

### Example 3: Administration of Eflapegrastim to Humans with

Docetaxel/Cyclophosphamide induced Neutropenia After 0.5 Hours Eflapegrastim 13.2 mg/0.6 mL (3.6 mg G-CSF) fixed dose is administered subcutaneously at 0.5 hours (±5 minutes) from the end of administration of Docetaxel 75 mg/m² IV, cyclophosphamide 600 mg/m² IV infusion time per institution's standard of care (“SOC”) to patients with early-stage breast cancer.

### Example 4: Administration of Eflapegrastim to Humans with Docetaxel/Cyclophosphamide Induced Neutropenia after 3 Hours

Eflapegrastim 13.2 mg/0.6 mL (3.6 mg G-CSF) fixed dose is administered subcutaneously at 3 hours (±15 minutes) from the end of administration of Docetaxel 75 mg/m² IV, cyclophosphamide 600 mg/m² IV (infusion time per institution's SOC) to patients with early-stage breast cancer.

### Example 5: Administration of Eflapegrastim to Humans with Docetaxel/Cyclophosphamide Induced Neutropenia after 5 Hours

Eflapegrastim 13.2 mg/0.6 mL (3.6 mg G-CSF) fixed dose is administered subcutaneously at 5 hours (±15 minutes) from the end of administration of Docetaxel 75 mg/m² IV, cyclophosphamide 600 mg/m² IV (infusion time per institution's SOC″) to patients with early-stage breast cancer.

### Example 6: Study of the Duration of Severe Neutropenia after the Same-Day, Varying Dosing Time Schedules of Eflapegrastim Administration in Patients with Breast-Cancer Receiving Docetaxel and Cyclophosphamide

The duration of Grade 4 neutropenia (absolute neutrophil count (ANC)<0.5×10⁹/L) is evaluated after treatment cycle 1.

In addition the following is evaluated:
the proportion of patients with Grade 4 neutropenia (ANC <0.5×10⁹/L) in treatment cycle 1
the time to recovery from severe neutropenia to a ANC ≥1.5×10⁹/L in treatment cycle 1
the incidence of Grade 3 febrile neutropenia in treatment cycle 1 (ANC <1.0×10⁹/L and either 1) a single temperature of >38.3° C. (101.0° F.) or 2) a sustained temperature of ≥38.0° C. (100.4° F.) for more than 1 hour
the pharmacokinetics (PK) of Eflapegrastim in treatment cycle 1
the incidence of neutropenic complications, including anti-infective use and hospitalizations due to neutropenia in patients during treatment cycle 1
the safety the Eflapegrastim treatment regimen
peripheral blood CD34⁺ counts

The same day dosing of Eflapegrastim, using a fixed dose of 13.2 mg/0.6 mL (3.6 mg G-CSF), is administered subcutaneously (SC) at varying dosing time schedules after administering docetaxel and cyclophosphamide (TC) to patients with early-stage breast cancer.

### Treatment Cycle 1

On day 1 of cycle 1, TC administration is followed by administration of the fixed dose of Eflapegrastim at one of the following time points proceeding the end of TC administration: 0.5 hours (±5 minutes), 3 hours (±15 minutes), and 5 hours (±5 minutes).

Prior to TC administration, patients may receive premedications for chemotherapy prophylaxis according to institutional standards of care (SOC). Intravenous (IV) administration of TC on Day 1 of each treatment cycle is as follows:
docetaxel 75 mg/m² IV, infusion time per institution's SOC;
cyclophosphamide 600 mg/m² IV, infusion time per institution's SOC;
docetaxel and cyclophosphamide dose modifications during cycle 1 are not permitted.

Up to 45 patients are enrolled in the study and randomized to one of the three Eflapegrastim dosing time schedules listed above using a 1:1:1 ratio in the study.

Blood for complete blood count (CBC) and pharmacokinetic (PK) analysis is drawn before the TC dose on Day 1 and post Eflapegrastim dose at 1 hour (±15 min), 3 hours (±15 min), 6 hours (±15 min), 8 hours (±15 min), 24 hours (±2 hours), 48 hours (±2 hours), 72 hours (±2 hours), 144 hours (Day 7±1 day) and 192 hours (Day 9±1 Day), and on Cycle 2, Day 1 (Day 22) before the TC dose. CBC analysis is performed by a clinical laboratory.

### Additional CBC Samples

In treatment cycle 1 only, CBC samples are drawn daily from Day 4 to Day 10. If on Day 10 the ANC is ≤1.0×10⁹/mL, CBC samples are drawn daily until the ANC is ≥1.5×10⁹/mL.

Peripheral blood CD34⁺ in Cycle 1

Peripheral blood CD34⁺ count samples are drawn from Day 2 to Day 10.

### Safety Visit for Cycle 1

On treatment cycle 2, day 1 (Day 22) all required assessments/evaluations are performed before TC administration for treatment cycle 2.

### Interim Safety Evaluation

A safety evaluation is conducted once the first 3 patients in each Eflapegrastim dosing time schedule have completed treatment cycle 1 of the study (total 9 patients). The safety evaluation includes adverse events (AEs), ANC and white blood cell (WBC) counts, duration of severe neutropenia (DSN) and neutropenic complications (hospitalization due to neutropenia, febrile neutropenia, use of anti-infectives).

After completing the safety evaluation the first 3 patients in each Eflapegrastim dosing time schedule, patients are enrolled to the different Eflapegrastim dosing time schedule as randomized if there are no safety findings in any of the three Eflapegrastim dosing time schedules. If it is determined from the safety review that one or more Eflapegrastim dosing time schedules are required to be stopped, all newly enrolled patients are re-randomized into the continuing Eflapegrastim dosing time schedules.

### Stopping Rules

Safety is evaluated in the first 3 patients in each Eflapegrastim dosing time schedule during treatment cycle 1. Further enrollment in a Eflapegrastim dosing time schedule is stopped when one of the following criteria is met:
1) 2 of 3 patients report febrile neutropenia in treatment cycle 1 and/or any Eflapegrastim-related Grade 4 AE
2) 2 of 3 patients report Grade 4 neutropenia and DSN is >2 days

Safety is monitored on an ongoing basis. Subsequent to the interim safety monitoring, a cohort is stopped for enrolling if a total of 3 or more patients (cumulative in a cohort) experienced febrile neutropenia (FN).

### Cycles 2 to 4

Eflapegrastim 13.2 mg/0.6 mL (3.6 mg G-CSF) is administered within 24 hours from the end of TC administration in all Eflapegrastim dosing time schedules. Patients must have an ANC ≥1.5×10⁹/L and platelet count ≥100×10⁹/L to begin each of the next cycles of chemotherapy. Patients are followed for safety. Each cycle is 21 days.

Blood samples for CBC in treatment cycles 2 to 4 are drawn on day 1 of each treatment cycle before chemotherapy and follow the SOC per cycle. CBC is drawn at the end-of-study visit 35 (±5) days after the last dose of study treatment (TC or Eflapegrastim).

### Duration of Study

Screening Period: Up to 30 days.

Treatment Period: Up to 4 treatment cycles (21 days per treatment cycle).

Safety Follow up Visit for Treatment Cycle 1: on treatment cycle 2, day 1 (day 22) before TC administration

End of Study Visit: 35 (±5) days after the last dose of study treatment (TC or Eflapegrastim)

### Inclusion Criteria

Patient must be willing and capable of giving written Informed Consent and must be able to adhere to Eflapegrastim dosing time administration, blood draw schedules, and meet all other study requirements.

Patient must have a new diagnosis of histologically confirmed early-stage breast cancer (ESBC), defined as operable Stage I to Stage IIIA breast cancer.

Patient must be a candidate to receive adjuvant or neoadjuvant TC chemotherapy.

Patient (male or female) must be at least 18 years of age.

Patient must have adequate hematological, renal, and hepatic function as defined by:
ANC ≥1.5×10⁹/L
Platelet count ≥100×10⁹/L
Hemoglobin ≥10 g/dL
Calculated creatinine clearance >50 mL/min
Total bilirubin ≤1.5 mg/dL
Aspartate aminotransferase (AST)/serum glutamic-oxaloacetic transaminase (SGOT) and alanine aminotransferase (ALT)/serum glutamic-pyruvic transaminase (SGPT)≤2.5×ULN, and alkaline phosphatase ≤2.0×ULN

Patient must have an Eastern Cooperative Oncology Group (ECOG) performance status ≤2.

Eflapegrastim is supplied in a sterile, single-use, pre-filled syringe with Eflapegrastim 13.2 mg/0.6 mL (3.6 mg G-CSF) administered SC. Eflapegrastim dose modification is not permitted.

### Assessments

The duration of Grade 4 neutropenia (ANC <0.5×10⁹/L) in treatment cycle 1 is evaluated.

The proportion of patients with Grade 4 neutropenia (ANC <0.5×10⁹/L) in treatment cycle 1 is evaluated

The time to recovery of severe neutropenia to ANC ≥1.5×10⁹/L in treatment cycle 1 is evaluated.

The incidence of Grade 3 febrile neutropenia in treatment cycle 1 (ANC <1.0×10⁹/L) and either a single temperature of >38.3° C. (101.0° F.) or a sustained temperature of ≥38.0° C. (100.4° F.) for more than 1 hour is evaluated.

The pharmacokinetics (PK) of Eflapegrastim in treatment cycle 1 is evaluated.

The incidence of Neutropenic Complications, including anti-infective use and hospitalizations due to neutropenia in patients during treatment cycle 1 is evaluated.

Peripheral blood CD34⁺ count is evaluated.

### Pharmacokinetic Assessments

Each patient starts chemotherapy on day 1 followed by fixed dose of Eflapegrastim administration timing based on each Eflapegrastim dosing time schedule. Blood samples for pharmacokinetic measurements and CBC are collected at:

### Cycle 1 Day 1

Pre-dose (before TC administration).

1, 3, 6, and 8 hours (±15 min) from Eflapegrastim dose time.

24, 48, and 72 (±2 hours) from Eflapegrastim dose time on day 1.

144 hours (Day 7±1 day) and 192 hours (Day 9±1 Day), from Eflapegrastim dose time on day 1.

### Cycle 2 Day 1

Before TC administration.

### Additional CBC Samples

In Cycle 1 only, CBC is also drawn daily from Day 4 to Day 10. If on Day 10 the ANC is ≤1.0×10⁹/L, CBC is drawn daily until the ANC is ≥1.5×10⁹/L.

### Peripheral Blood CD34⁺ Counts in Cycle 1

Peripheral blood CD34⁺ counts are drawn daily from day 2 to day 10.

### Safety Assessments

Safety is assessed throughout the study by reported/elicited AEs, laboratory assessments, and physical examinations.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art is explicitly excluded from any one or more of the claims. Because such embodiments are deemed to be known to one of ordinary skill in the art, they is excluded even if the exclusion is not set forth explicitly herein. Any particular embodiment of the disclosure can be excluded from any claim, for any reason, whether or not related to the existence of prior art.

### EQUIVALENTS

The invention is embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

LISTING OF VARIOUS SEQUENCES
SEQ ID NO: 1
TPLGPASSLPQSFLLKCLEQVRKIQGDGAALQEKLATYKLCHPEELVLLG
HSLGIPWAPLSSCPSQALQLAGCLSQLHSGLFLYQGLLQALEGISPELGP
TLDTLQLDVADFATTIWQQMEELGMAPALQPTQGAMPAFASAFQRRAGGV
LVASHLQSFLEVSYRVLRHLAQP
SEQ ID NO: 2
TPLGPASSLPQSFLLKSLEQVRKIQGDGAALQEKLCATYKLCHPEELVLL
GHSLGIPWAPLSSCSQALQLAGCLSQLHSGLFLYQGLLQALEGISPELGP
TLDTLQLDVADFATTIWQQMEELGMAPALQPTQGAMPAFASAFQRRAGGV
LVASHLQSFLEVSYRVLRHLAQP
SEQ ID NO: 3
PSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCS
VMHEALHNHYTQKSLSLSLGK
SEQ ID NO: 4
PSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCS
VMHEALHNHYTQKSLSLSLGK

A sequence listing electronically submitted with the present application on Dec. 7, 2020 as an ASCII text file named 20201207_Q23319LM01_TU_SEQ, created on Dec. 7, 2020 and having a size of 8,000 bytes, is incorporated herein by reference in its entirety.

What is claimed is:

1. A method for increasing an absolute neutrophil count, the number of granulocytes, stem cell production, hematopoiesis, the number of hematopoietic progenitor cells, or stem cell production, or for treating or preventing the condition characterized by compromised white blood cell production in a patient in need thereof, the method comprising administering an effective amount of Eflapegrastim within a period of less than 24 hours after the patient is administered a chemotherapeutic agent or receives a radiotherapy.

2. The method of claim 1, wherein the condition characterized by compromised white blood cell production is selected from the group consisting of chemotherapy-induced neutropenia, radiotherapy-induced neutropenia, reduced hematopoietic function, reduced immune function, reduced neutrophil count, reduced neutrophil mobilization, mobilization of peripheral blood progenitor cells, sepsis, bone marrow transplants, infectious diseases, leucopenia, thrombocytopenia, anemia, enhancing engraftment of bone marrow during transplantation, enhancing bone marrow recovery in treatment of radiation, chemical or chemotherapeutic induced bone marrow aplasia or myelosuppression, radiotherapy-induced bone marrow aplasia or myelosuppression, and acquired immune deficiency syndrome

3. The method of claim 1, wherein the condition is a chemotherapy-induced neutropenia or a radiotherapy-induced neutropenia.

4. The method of claim 3, wherein the method reduces the duration of chemotherapy-induced neutropenia or radiotherapy-induced neutropenia in a patient in need thereof.

5. The method of claim 1, wherein the method comprises administering an effective amount of Eflapegrastim on the same day when the patient is administered a chemotherapeutic agent or receives radiotherapy.

6. The method of claim 1, wherein administering the effective amount of Eflapegrastim reduces the duration of an absolute neutrophil count of less than about 0.5×10⁹/L in the patient to less than about 6 hours, about 12 hours, or 24 hours.

7. The method according to claim 6, wherein administering the effective amount of Eflapegrastim prevents the absolute neutrophil count in the patient from reaching less than about 0.5×10⁹/L.

8. The method of claim 1, wherein upon administration of the effective amount of Eflapegrastim, an absolute neutrophil count of the patient increases from the first occurrence of less than about 0.5×10⁹/L to greater than or equal to about 1.5×10⁹/L within less than about four days, about seven days, or about ten days.

9. The method of claim 1, wherein the effective amount of Eflapegrastim is administered concomitantly with the chemotherapeutic agent or the radiotherapy.

10. The method of claim 1, wherein the effective amount of Eflapegrastim is administered within about 0.5 hours, about 3 hours, or about 5 hours after the administration of the chemotherapeutic agent or the receipt of the radiotherapy.

11. The method of claim 1, wherein the effective amount of Eflapegrastim is administered within about 0.5 hours, about 1 hour, about 2 hours, about 3 hours, about 4 hours, about 5 hours, about 6 hours, about 7 hours, about 8 hours, about 9 hours, about 10 hours, about 11 hours, or about 12 hours after the administration of the chemotherapeutic agent or the receipt of the radiotherapy.

12. The method of claim 5, wherein the chemotherapeutic agent is a myelosuppressive chemotherapeutic agent.

13. The method claim 12, wherein the myelosuppressive chemotherapeutic agent is selected from the group consisting of docetaxel, cyclophosphamide, doxorubicin, etoposide, cisplatin, paclitaxel, topotecan, vincristine, methylprednisolone, cytarabine, and combinations thereof.

14. The method of claim 1, wherein the patient is receiving the chemotherapeutic agent or the radiotherapy to treat a cancer selected from the group consisting of breast cancer, non-small cell lung cancer, small cell lung cancer, ovarian cancer, sarcoma, urothelial cancer, germ cell tumors and non-Hodgkin's lymphoma.

15. The method of claim 1, wherein administering an effective amount of Eflapegrastim comprises administering parenterally from about 2 to 18 mg of Eflapegrastim.

16. The method of claim 15, wherein administering an effective amount of Eflapegrastim comprises administering parenterally about 13.2 mg of Eflapegrastim.
